(19) United States
(12) Patent Application Publication
(10) Pub. No.: US 2004/0234972 A1
(43) Pub. Date: Nov. 25, 2004

(54) Method for identifying and purifying smooth muscle progenitor cells

(76) Inventors: Gary K. Owens, Earlysville, VA (US); Ichiro Manabe, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/472,414

(22) PCT Filed: Mar. 19, 2002
(86) PCT No.: PCT/US02/08402

Related U.S. Application Data
(60) Provisional application No. 60/277,202, filed on Mar. 20, 2001.

(51) Int. Cl. C12Q 1/68; C12N 5/08; C12N 15/85

(52) U.S. Cl. 435/6; 435/455; 435/366

(57) ABSTRACT

The present invention relates to purified smooth muscle progenitor cells and a method for isolating such cells. The purified smooth muscle progenitor cells of the present invention are capable of being induced into the smooth muscle cell lineage at high efficacy (i.e. greater than 60% conversion). The method comprises the steps of transforming cell populations that contain totipotent or pluripotent cells with DNA constructs that are expressed only in the smooth muscle cell lineage, inducing a portion of those cells and identifying those cells that express the construct only after the cells are induced.

### RELATED APPLICATION

This application claims priority under 35 USC §119(e) to U.S. Provisional Application Serial No. 60/277,202, filed Mar. 20, 2001, the disclosure of which is incorporated herein.

### US GOVERNMENT RIGHTS

This invention was made with United States Government support under Grant Nos. P01 HL19242, R01 HL38854 and R37 HL57353, awarded by the National Institutes of Health. The United States Government has certain rights in the invention.

### FIELD OF THE INVENTION

The present invention is directed to stem cells and methods of preparing populations of progenitor cells that differentiate into a preselected cell type with high efficiency.

### BACKGROUND OF THE INVENTION

There is currently extensive interest in developing methods for using pluripotential stem cell populations for a wide variety of potential therapeutic applications including delivery of therapeutic genes, correction of gene defects, replacement/augmentation of existing dysfunctional cell populations (e.g. dopaminergic neurons in Parkinsons Disease), and generation of organs/tissues for surgical repair/replacement. However, existing methods in the field have a number of major limitations that relate to obtaining purified population s of the desired cell types from pluripotent stem cells.

First of all existing methods are relatively inefficient in producing the desired cell type, and/or result in production of mixed populations of cells including many undesired contaminants. Second, current methods often result in the production of cells that have lost many of the desired characteristics needed for effective therapeutic uses including the ability to effectively invest tissues/organs in vivo (e.g. systems in which only terminally differentiated not precursor cells are produced). Third, existing methodologies of isolating stem cells cannot be applied to somatic pluripotential stem cells and thus require the use of heterologous stem cells (i.e. from another individual, thus posing major immune complications) or the availability of cryopreserved embryonic stem cells from umbilical chord specimens (available for a very limited number of individuals). Fourth, the lack of sufficient knowledge regarding what factors/environmental cues are needed to induce specification of desired cell lineages has also greatly hindered the ability to produce desired cell types from stem cells. Finally, the current techniques are extremely expensive due to the complexity of purified reagents and growth factors needed to induce desired cell lineages.

The present invention provides a method of identifying pluripotent cell populations that differentiate into a preselected cell type with high efficacy. The pluripotent progentitor cell populations are identified and purified through the use of reporter gene constructs that are only expressed in the desired cell type. For example, in one embodiment smooth muscle progenitor cells are isolated and purified by transforming a population of pluripotent cells with a DNA construct comprising a smooth muscle promoter operably linked to a marker.

A large number of major human diseases including coronary artery disease, hypertension, and asthma are associated with abnormal function of smooth muscle cells (SMC). In addition, SMC dysfunction also contributes to numerous other human health problems including vascular aneurysms, and reproductive, bladder, and gastrointestinal disorders. It is estimated that over $250 billion dollars in annual health care costs in the USA alone are related to pathologies associated with the SMC. One unique embodiment of the methodology described herein is the production of SMC or SMC progenitor cells from various human multi-potential stem cell populations for use in a variety of potential clinical applications. This includes but is not limited to the following:

1. In vitro production of SMC tissues or cells for surgical repair or augmentation in vivo (e.g. augmentation of bladder or gastrointestinal function; repair of vascular aneurysms; stabilization of atherosclerotic plaques; repair of traumatic injuries to SM tissues; repair/regeneration of SMC organs/tissues after surgical resection of a tumor; surgical correction of congenital abnormalities in SMC tissues; vascular coronary bypass, repair of vascular malformations, etc.). One embodiment involves use in tissue engineering in vitro and subsequent use for surgical repair/augmentation. Other uses involve simple augmentation of existing tissues with stem cell derived SMC or SMC progenitors, or stem cell derived SMC tissues.

2. Delivery of therapeutic genes for treatment of SMC related diseases such as atherosclerosis, asthma, hypertension, etc. In this embodiment of the technology, stem cell derived SM tissues or cells would be genetically engineered to express a desired therapeutic gene or agent and surgically implanted into a desired treatment site in vivo. An example would be implantation of stem cell derived vascular SMC that express high levels of NO synthase into coronary vessels as a means of treating coronary atherosclerosis or re-stenosis.

3. Correction of gene mutations that contribute or cause SMC related diseases. In this aspect of the technology, one would replace defective genes with normal genes within the stem cells, and then isolate and purify stem cell derived SMC populations for augmentation therapies. An example of an application of this type would be for Marfan's syndrome a disease caused by mutations in the fibrillin gene that encodes for an extracellular matrix protein important for skeletal muscular development, and for stability of blood vessels. The most frequent cause of death in these patients is vascular aneurysm, and there are no effective therapies or cures. Stem cells would be derived from bone marrow or other source from the Marfan's patient, the defective fibrillin gene would be replaced with a normal one using techniques standard for experts in the field. These stem cells would then be induced to form SMC lineages and purified using the techniques claimed herein and purified stem cell derived SMC or SMC progenitors implanted into blood vessels.

4. Promotion of vascular development as part of efforts to produce autologous organs in vitro for organ replacement surgery. Organogenesis is critically dependent on development of a vascular supply. Moreover, SMC are the major cell type present in many organs including stomach, intestine, uterus, bladder, etc. As such, the methodologies described may have important applications in efforts to generate these organs for organ transplantation/organ augmentation therapies.

Origins of SMC and Molecular Control of SMC Lineage Determination:

The developmental program for SMC is poorly understood. Whereas this program must reflect the multifunctional role of SMC in development, controversy exists regarding which cell populations have the capacity to differentiate into SMC, when during embryological development multi-potential cells commit to a SMC lineage, and what criteria define a “committed” SMC. There have been unsubstantiated claims in the literature that virtually any “mesodermal” cell may have the capacity to differentiate into SMC when appropriately stimulated. However, such claims appear to be inconsistent with observations that development of SMC during embryogenesis is tightly regulated within specific spatial-temporal domains, that many mesodermal cells fail to form SMC lineages despite their close proximity to such domains, and that certain SMC subtypes are derived from distinct embryological origins. For example, the vascular SMC of the great vessels (ascending aorta and branchial arches) are derived from the cranial neural crest, while vascular SMC of the coronary circulation are derived by epithelial-mesenchymal transformation of epicardial cells. There is even evidence, albeit controversial, that certain populations of endothelial cells may trans-differentiate into vascular SMC.

In the final analysis, it is likely that lineage determination in SMC, as with other cell types, results from the complex interplay of environmental factors (or cues), and genetic programs that control the pattern of gene expression appropriate for a given cell type. It has been useful to experimentally define the process of cell lineage determination as consisting of a finite number of discreet steps such as “specification”, “determination”, and “commitment”. However, although there are clear conceptual and chronological differences between commitment and the earlier decisions resulting in cell specification and determination, there may be no fundamental molecular difference between these types of events, i.e. they may represent a continuous series of events acting on different sets of genes whose gene products in turn further and further limit the developmental potential of a given cell. A key challenge for the vascular biology field has been to define the events, factors, and molecular processes whereby primordial cells ultimately give rise to fully differentiated SMC.

There has been considerable progress in recent years in defining the molecular processes and environmental factors that control late stages of SMC differentiation. However, a major limitation in the field has been the lack of an inducible lineage system with which to study the earliest stages of differentiation of SMC from pluri-potential embryonic stem cell populations. As a consequence, virtually nothing is known regarding the molecular genetic determinants of lineage in SMC. To overcome limitations of existing SMC culture systems, several groups have developed in vitro culture systems in which multi-potential cells, including mouse embryonal carcinoma cells (P19) (Blank, et al., J.Cell.Biochem. 17D:218 (1993)), neural crest stem cells (Monc-1) (Jain, et al., The Journal of Biological Chemistry 273(11), 5993-5996 (1998)), mouse embryonic stem cells (Drab, et al., Faseb Journal 11:905-915 (1997), mouse embryonic 10T1/2 cells (Hirschi, et al., J. Cell Biol. 141:805-814 (1998)), and chick proepicardial cells (Landerholm, et al., Development 126:2053-2062 (1999)), are induced to differentiate into SMC or SMC-like cells. However, these systems all have major limitations including: low efficacy and efficiency of conversion to SMC, an extremely long time lag between “induction” of SMC lineage and the availability of purified populations of cells to study, poor efficacy of induction of definitive SMC marker genes such as SM MHC, induction of SMC that are incompletely differentiated (e.g. lack the ability to contract), technical difficulties in isolating and/or maintaining cells in a multi-potential state, lack of control over induction of lineage conversion/differentiation, and/or uncertainties regarding the original embryological origins of the multi-potential cells. The invention described herein circumvents each these limitations and for the first time permits high throughput screening, identification, and purification of SMC or SMC “progenitor” cells from multi-potential stem cell populations. Moreover, of critical importance, the invention described is potentially adaptable for use with virtually any source of multi- or totipotent cells.

The approach for production of stem cell derived SMC and SMC progenitors described in the present invention is based on the use of the unique SMC promoter-enhancers described in International Applications PCT/US9901038, PCT/US99/24972, and U.S. Provisional Application No. 60/263,811 in combination with unique methodologies, and several described in the prior art, including use of an embryoid body model for induction of SMC and other cell lineages (Drab, et al., Faseb Journal 11:905-915 (1997) and Keller, G. M. Curr. Opin. Cell Biol. 7:862-869 (1995)).

Embryonic stem cells exhibit nearly unlimited renewal capacity while being able to maintain a pluripotential state and so possess tremendous potential in a wide variety of tissue engineering applications. Cultivation of ES cells in aggregates, known as embryoid bodies, is required in order for them to display their full differentiation capacity in vitro (Keller, G. M. Curr. Opin. Cell Biol. 7:862-869 (1995)). As embryoid bodies, these cells recapitulate many of the events of early embryonic development, including development of the three embryonic germ layers and have the potential to form a wide variety of differentiated cell types.

To our knowledge, the embryoid body model is the only one in which fully contractile SMC are formed de novo in culture. Moreover, the system has been shown to work with multiple pluripotential stem cell sources including those from human (Itskovitz-Eldor, et al., Mol.Med. 6:88-95 (2000) and Schuldiner, et al., Proc.Natl.Acad.Sci.USA. 97:11307-11312 (2000)) and is likely to be adaptable for any pluripotential stem cell source. This is important for any potential commercial application geared towards using an individual's own stem cells for therapeutic purposes. Several other cell model systems have been used to explore control of early stages of specification of SMC including multipotential cells such as 10T1/2, and neural crest stem cells derived from mice. However, a limitation of these models is that the SMC-like cells derived fail to express a number of key SMC differentiation markers, and cells do not exhibit contractile ability. That is, these systems fail to produce fully differentiated SMC presumably due to the inability to recapitulate the complex environmental cues necessary for this process. Moreover, the latter cell systems have no potential use in man since they represent unique mouse cell lines.

It is known that developing SMC, like most other developing cell types, are highly sensitive to and regulated by local environmental signals. A major strength of the embryoid body model used as part of the current invention is that it allows heterotypic cell-cell and cell-matrix interactions and growth factor mediated signaling in a way that mimics the embryonic milieu. Thus, SMC develop under optimal conditions for the formation of mature, fully functional cells. By contrast, it is probable that other in vitro model systems of “SMC” development are not able to recapitulate many of the cues present in vivo, and such models may as a result only undergo part of the SMC developmental program. Accordingly, these systems only express a subset of smooth muscle specific genes, while lacking other essential components of the developmental program that would enable the formation of fully functional tissue. Whereas the embryoid body itself has many unique advantages, by itself it has virtually no potential commercial utility, since its strength, the induction of multiple cell lineages without use of complex lineage inducing agents, is also its main limitation. That is, the embryoid body model produces a multitude of different cell types and a relatively small fraction of a particular cell type (typically <5%). Although one can enrich for a particular cell type by treatment with various inducing agents, at best one can achieve only enriched populations of cell types of interest with >80% contaminating cells.

The methods described in the present invention are unique in that they are the first that permit high efficiency production and purification of SMC or SMC progenitors from pluripotential or totipotential stem cells. Moreover, this experimental approach has a number of additional major advantages over existing technologies with respect to potential therapeutic applications in humans including:

a) Methods are adaptable for use with a variety of different sources of totipotential or pluri-potential somatic stem cell populations including those derived from bone marrow (Ferrari, et al., S 279:1528-1530 (1998)), umbilical vessels, and adipose tissue (Zuk, etal., Tissue Engineering 7:211-228 (2001)). That is cells can readily be derived from an individual's own stem cells, a huge advantage for potential therapeutic applications since it will eliminate immune complications common with other technologies using heterologous stem cell sources.

b) Stem cell derived SMC are likely to retain much greater potential for forming (or integrating into) complex tissues and organs as compared to SMC derived from pre-existing smooth muscle tissues.

c) Stem cells can be easily genetically manipulated and expanded to generate the number of cells required.

d) SM MHC subtype specific promoters-enhancers previously identified and described in Manabe, I. and Owens, G. K. J.Biol.Chem. 276:39076-39087 (2001) and Manabe,I. and Owens, G. K. J.Clin.Invest. 107:823-834 (2001)) will enable the present methods to be adapted for producing specific subtypes of SMC including vascular, airway, intestinal, and uterine SMC from multiple somatic stem cell sources.

e) The use of embryoid bodies in induction of cell lineages including SMC eliminates the need for complete knowledge of the complex array of environmental cues necessary for induction of cell lineages, and extensive use of expensive purified growth factors and lineage inducing agents that may or may not be available (or known).

While the methods described herein have been exemplified for the isolation of SMC and smooth muscle progenitor cells, they are readily adaptable to the production of any desired cell type simply by replacing the SMC specific/selective promoter/enhancer of the reporter gene construct (used in identifying and purifying the progenitor cells) with an appropriate promoter regulatory element that is selective/specific for the cell type of interest. Examples include the use of promoter/enhancers specific for, cardiac myocytes (Sah et al., J. Clin. Invest. 103: 1627-1634 (1999)), endothelial cells (Schlaeger et al., Development 121: 1089-1098 (1995)) and neurons (Miyachi et al., Molecular Brain Research 69:223-231(1999))

### SUMMARY OF INVENTION

The present invention is directed to a method for identifying, and purifying a unique population of pluripotential progenitor cells that can be induced to form specific preselected cell type lineages with extremely high efficacy. The present invention also provides a method for preparing autogenous populations of cells of one specific cell type, from the totipotent or pluripotent cells of an individual.

In addition, the invention defines a unique combination of new and pre-existing methods that permit production and purification of SMC or SMC progenitor cells derived from various embryonic or somatic stem cell populations. Finally the methodology permits isolation and purification of stem cell derived SMC or SMC progenitors specific for a particular subtype of SMC including but not limited to vascular, intestinal, uterine, airway or bladder SMC.

### BRIEF DESCRIPTION OF THE DRAWINGS

FIG. 1 is a flow chart showing the steps for isolating SMC progenitor cells from various stem cell sources.

FIG. 2 is a schematic representation of the protocol used to induce SMC lineages in a representative pluripotential somatic stem cell system (i.e. A404 P19 embryonal carcinoma cells). Transfected cells were treated with retinoic acid (RA) for 3 days. On day 4 puromycin was added to the medium and cells were treated treated with puromycin for either 2 days or 5 days.

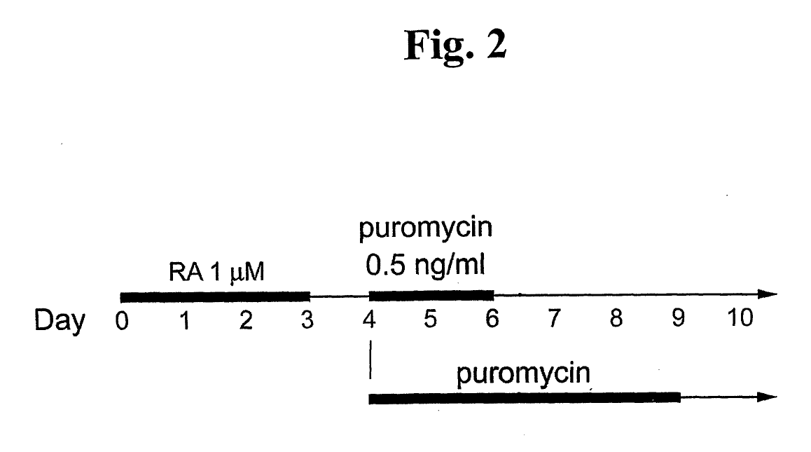

### DETAILED DESCRIPTION OF THE INVENTION

Definitions

In describing and claiming the invention, the following terminology will be used in accordance with the definitions set forth below.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

A “polylinker” is a nucleic acid sequence that comprises a series of three or more different restriction endonuclease recognition sequences closely spaced to one another (i.e. less than 10 nucleotides between each site).

As used herein, the term “vector” is used in reference to nucleic acid molecules that have the capability of replicating autonomously in a host cell, and optionally may be capable of transferring DNA segment(s) from one cell to another. Vectors can be used to introduce foreign DNA into host cells where it can be replicated (i.e., reproduced) in large quantities. Examples of vectors include plasmids, cosmids, lambda phage vectors, viral vectors (such as retroviral vectors).

A plasmid, as used herein, is a circular piece of DNA that has the capability of replicating autonomously in a host cell. A plasmid typically also includes one or more marker genes that are suitable for use in the identification and selection of cells transformed with the plasmid.

As used herein a “gene” refers to the nucleic acid coding sequence as well as the regulatory elements necessary for the DNA sequence to be transcribed into messenger RNA (mRNA) and then translated into a sequence of amino acids characteristic of a specific polypeptide.

A “marker” is an atom or molecule that permits the specific detection of a molecule comprising that marker in the presence of similar molecules without such a marker. Markers include, for example radioactive isotopes, antigenic determinants, nucleic acids available for hybridization, chromophors, fluorophors, chemiluminescent molecules, electrochemically detectable molecules, molecules that provide for altered fluorescence-polarization or altered light-scattering and molecules that allow for enhanced survival of an cell or organism (i.e. a selectable marker). A reporter gene is a gene that encodes for a marker.

A promoter is a DNA sequence that directs the transcription of a DNA sequence, such as the nucleic acid coding sequence of a gene. Promoters can be inducible (the rate of transcription changes in response to a specific agent), tissue specific (expressed only in some tissues), temporal specific (expressed only at certain times) or constitutive (expressed in all tissues and at a constant rate of transcription).

A core promoter contains essential nucleotide sequences for promoter function, including the TATA box and start of transcription. By this definition, a core promoter may or may not have detectable activity in the absence of specific sequences that enhance the activity or confer tissue specific activity.

An “enhancer” is a DNA regulatory element that can increase the efficiency of transcription, regardless of the distance or orientation of the enhancer relative to the start site of transcription.

As used herein, the term “purified” and like terms relate to the isolation of a molecule or compound in a form that is substantially free of contaminants normally associated with the molecule or compound in a native or natural environment. “Operably linked” refers to a juxtaposition wherein the components are configured so as to perform their usual function. Thus, promoters operably linked to a coding sequence are capable of effecting the expression of the coding sequence.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein the term “totipotent” or “totipotential” and like terms refers to cells that have the capability of developing into a complete organism or differentiating into any cell type of that organism.

As used herein the term “pluripotent” or “pluripotential” and like terms refers to cells that cannot develop into a complete organism, but retain developmental plasticity, and are capable of differentiating into some of the cell types of that organism.

As used herein a “differentiated cell type” refers to a cell that expresses gene products that are unique to that cell type. For example, a smooth muscle cell is a cell type that expresses specific markers associated with smooth muscle cells, including smooth muscle α-actin, smooth muscle myocin heavy chain (MHC), h1-calponin, and smoothelin.

As used herein a “progenitor cell” of a specified cell type is a cell that has the capacity to become the specified cell type. For example a smooth muscle progenitor cell does not express the specific markers associated with smooth muscle cells, but it has the capacity to differentiate into a cell that does express those markers.

As used herein the term “somatic stem cell” refers to pluripotent stem cells derived from various somatic tissue sources including bone marrow, adipose tissue, or tumor sources (i.e. P19).

The Invention

In accordance with one embodiment of the present invention a unique method for identifying and purifying specific progenitor cell populations is provided.

The method comprises the steps of transfecting a population of cells with a gene construct, wherein the population of cells comprises pluripotent or totipotent cells and the gene construct comprises an appropriate promoter operably linked to a marker. For the purposes of the present invention an appropriate promoter is any promoter that is selective/specific for the desired cell type (i.e. the promoter will express operably linked coding sequences only in the desired cell type). The promoter includes all the necessary regulatory elements to provide for optimal selective expression, and in one embodiment optimal cell/tissue specific expression requires the addition of an enhancer (i.e. a promoter/enhancer).

In accordance with one embodiment of the present invention a method of generating a substantially pure population of differentiated cells from a cell population comprising totipotent or pluripotent cells is provided. In preferred embodiments the substantially pure population of differentiated cells comprises greater than 90% of the desired cell type and more preferably greater than 95% of the desired cell type and most preferably a purity of 99% or 100% of the desired cell type. The method comprises the steps of transfecting a population of cells comprising totipotent or pluripotent cells with a nucleic acid gene construct comprising a promoter operably linked to a marker. The promoter (or promoter/enhancer) element functions only in the desired cell type, and thus the marker is expressed only in cells that have differentiated into the desired cell type. The transfected population of cells is then induced to differentiate into the desired cell type using techniques known to those skilled in the art and the cells expressing the marker are isolated.

The population of cells comprising totipotent or pluripotent cells can be isolated from a number of sources. More particularly, the cells can be isolated from umbilical tissue, adipose tissue, bone marrow of a mammal, including humans. When the differentiated cells are to be used for therapeutic purposes to treat an individual, preferably the cells will be isolated from the same individual to be treated with the differentiated cells (i.e. autogenous cells).

Procedures for inducing totipotent or pluripotent cells to differentiate into specific cell types have been described previously and are known to the skilled practitioner. In accordance with one embodiment, the formation of embryoid bodies is used to induce the differentiation of stem cells. Embryoid body formation and its use to induce stem cell differentiation has been described previously in the literature (Keller, G. M. Molecular & Cellular Biology 17: 2266-2278 (1997)). In one embodiment of the present invention SMC progenitor cells are isolated and are used to generate smooth muscle cells. Several methods have been previously described for converting SMC progenitor cells into SMC lineages. These methods vary depending on the type of multipotential cell line employed. For the somatic stem cell designated “P19” this involves treatment of monolayer cultures with retinoic acid, whereas for ES or somatic stem cells this involves aggregation of ES cells into embryoid bodies followed by treatment with retinoic acid plus dibutyryl cAMP (Blank et al., Circulation Research 76:742-749 (1995). Methods are also described in the literature for inducing and/or enriching for many other desired cell types including neurons and cardiomyocytes (Kehat et al., J. Clin. Invest. 108: 407-414 (2001); Weiss et al., J. Clin. Invest. 97: 591-595 (1996); and Zhang et al., Nature Biotechnology 19: 1129-1133 (2001)). One can vary the length of the induction period to isolate differentiated, mature, or precursor populations of the desired cell type.

One of the key elements of the present invention is the transfection of the pluripotent and totipotent cell populations with a reporter gene construct that is only expressed in the desired cell type. In this manner the desired cells can be purified by screening or selecting for cells expressing the marker. Accordingly, the promoter or promoter/enhancer used in the reported gene construct is selected based on the cell type to be isolated. For example, if a smooth muscle cell type is desired, the construct will comprise a smooth muscle promoter/enhancer selected from the group consisting of smooth muscle α-actin (Mack and Owens, Circ. Res. 84: 852-861(1999)), SM22 (Kim et al., Molecular & Cellular Biology 17: 2266-2278 (1997)), calponin (Miano et al., Journal of Biological Chemistry 275(13), 9814-9822 (2000)), smoothelin (Rensen et al., Cytogenietics & Cell Genietics 89: 225-229 (2000)) and smooth muscle myosin heavy chain (Madsen et al., Circ. Res. 82: 908-917 (1998)) promoters.

The marker used in accordance with the present invention can be selected from any of the known visible or selectable markers that are biocompatible and known to the skilled practitioner. Preferably the marker will be one that allows for easy screening or more preferably allows for the selection or sorting of cells based on the expression of the reporter gene construct. In one embodiment, marker is a flourophore, such as the green fluorescent protein and desired cells are isolated by fluorescent activated cell sorting (FACS). Alternatively the maker may encode for selectable marker that allows for enhanced survival of an cell, (i.e. an antibiotic resistance gene). Advantageously, when the marker is a selectable marker, the desired differentiated cells are identified and simultaneously isolated by culturing the population of cells under conditions where only those cells expressing the marker survive.

In accordance with one embodiment the nucleic acid construct used to transfect the stem cells comprises a first gene construct comprising a constitutive promoter operably linked to a second marker and a second gene construct comprising a tissue/cell specific promoter operably linked to a first marker. The first gene construct allows for the identification of cells that have been successfully transfected with the nucleic acid construct. The second gene construct is expressed only in cells that have differentiated into the desired cell type and thus serves to identify the differentiated cells.

The present invention also provides a method of identifying the progenitor cells of a desired differentiated cell type. More particularly the present invention allows for the identification of pluripotent stem cell populations that will yield greater than 60% and more preferably greater than 80% of a preselected cell type upon induction of the pluripotent cell population. In accordance with one embodiment the present invention provides a population of pluripotent stem cells, and a method for preparing such cells, that yield greater than 90% and more preferably greater than 95% of a preselected cell type upon induction of the pluripotent cell population. The method of producing such populations of stem cells comprises transfecting a population of cells, that includes totipotent or pluripotent cells, with a nucleic acid sequence comprising a promoter/enhancer element that functions only in the desired cell type, wherein the promoter/enhancer element is operably linked to a marker. The transfected cells are then induced to become the desired cell type and the progenitor cells that gave rise to the desired cell type are then identified. In this embodiment the cells are induced to differentiate, but the inducing agent is removed before the cells are terminally differentiated.

In one embodiment the pluripotent or totipotent cells are induced to begin differentiating by forming an embryoid body from the cells. The cells are allowed to differentiate for a predetermined length of time, and in one embodiment, before the cells begin to express markers associated with the desired cell type, the cells are dissociated (and any other inducing agents removed) and individual cells or small clumps of 1-3 cells are isolated. The individual cells/small clumps of cells are then clonally propagated in the absence of further induction. A portion of each clonally propagated population of cells is then induced to determine which pluripotent cell populations will give rise to the desired terminally differentiated cell type. The pluripotent stem cells can be stored (i.e. frozen) for future use, further cultured under non-inducing conditions to clonally expand the population of cells and/or optionally pooled together before storing. Cells can also be easily genetically modified during this time using techniques known to those skilled in the art. This might include correction of a gene defect or insertion of a therapeutic gene. These pluripotent progenitor cells are anticipated to have greater viability during long storage than fully differentiated cells that have less developmental plasticity, or embryonic stem cells frozen within umbilical chord vessels.

Alternatively the cells of the embryoid body may be cultured under inducing conditions for a length of time sufficient to allow proliferation and differentiation of the cells. Substantially pure populations of the desired differentiated cell types can then be identified and recovered based on the expression of the reporter gene.

In accordance with one embodiment a method for identifying and purifying smooth muscle cell (SMC) progenitor cell populations is provided. The method comprises the use of SMC specific-selective promoter/enhancers such as SM α-actin and SM myosin heavy chain (described in International patent applications nos. PCT/US9901038 and PCT/US99124972, respectively, the disclosures of which are expressly incorporated herein) for selection, identification, and screening of candidate SMC progenitor populations in mice or other species. In addition promoter/enhancer constructs have been described that display SMC subtype selectivity (see U.S. Provisional Application No. 60/263,811, the disclosure of which is incorporated herein). These SMC subtype promoter/enhancer elements can also be used in accordance with the present invention.

Prior to the present invention there was no known methods for purifying SMC progenitor populations from pluripotential embryonic cells, or tissue samples. Although a number of cell systems have been described in which multipotential cells can be induced to form SMC lineages in vitro, previous studies did not define either methods or cell lines that would permit purification of relatively pure populations of SMC progenitor cells. Rather, each of these publications focused on how multipotential cells could be induced to differentiate into SMC. In addition, the present invention is the first to describe a procedure for isolating SMC progenitor populations from human tissues and cells. Previously described systems relied on use of existing established cell lines, and focused on studies of SMC differentiation but not the earlier determination events that control SMC lineage. There were also additional major practical limitations with each of these previously described SMC differentiation systems that preclude their practical use for isolating SMC progenitor cells from human samples. These limitations include: a very low efficiency of conversion of multipotential cells to SMC (<1-5%), poor induction of SMC markers, production of SMC that are incompletely differentiated, lack of control of induction of SMC markers, and use of a cell sources unavailable in humans (e.g. the pro-epicardial organ or neural crest cells, poor reproducibility/technical difficulties in growing cells [e.g. use of chick embryo extract] and insufficient knowledge or availability of required lineage inducing factors.

In accordance with one embodiment, greater than 50% of the pluripotent cells can be induced to express multiple SMC differentiation marker genes. In one embodiment, >90% of the pluripotent smooth muscle progenitor cell line isolated from mouse A404 cells can be induced to express multiple SMC differentiation marker genes including the definitive SMC lineage marker smooth muscle myosin heavy chain (SM MHC) by treatment with retinoic acid. Similarly, other pluripotent progenitor cell lines have been isolated that show high efficacy (i.e. greater than 60% conversion, more preferably greater than 80% conversion) of commitment to a SMC lineage upon retinoic acid treatment, thus demonstrating the reproducibility of the present methodology. In accordance with one embodiment, the present invention is directed to the production and purification of human SM progenitor cells from various totipotential or pluripotential cell systems including, but not limited to embryonic stems cells, or somatic stem cells derived from bone marrow, adipose tissue, or embryonal carcinoma cells.

In one embodiment of the present invention, a method of identifying smooth muscle progenitor cells is provided. The method comprises the steps of transfecting a population of cells that includes totipotent or pluripotent cells with a nucleic acid sequence comprising a smooth muscle cell specific promoter/enhancer operably linked to a marker. The population of cells is then induced to differentiate, and smooth muscle progenitor cells based on the expression of the marker. In one embodiment an embryoid body is formed from the progenitor cells and the cells are allowed to begin to differentiate. In addition to formation of the embryoid body the cells can be further induced by the addition of retinoic acid and/or dibutyryl cAMP.

In one embodiment the cells of the embryoid body are dissociated at a developmental stage where the cells remain pluripotent, and individual cells are clonally propagated to generate pools of progenitor cells. A portion of each clonally propagated pluripotent cell is stored, while the remaining portion is allowed to differentiate to a developmental stage wherein smooth muscle cell specific genes are expressed. These differentiated cells are then screened or selected for cells that express the marker. Those clonal populations of cells that differentiate into primarily (i.e. greater than 60% and more preferably greater than 90%) into smooth muscle cells indicate that the corresponding parental pool of clonally propagated puripotent cell are in fact smooth muscle progenitor cells.

In accordance with one embodiment, the method for isolating SMC progenitor cells comprises the steps of transfecting pluripotential cells such as somatic stem cells, P19 embryonal carcinoma cells, or embryonic stem cells with a selectable marker gene operably linked to the SM α-actin or SM myosin heavy chain promoter/enhancer described in International patent applications nos. PCT/US9901038 and PCT/US99/24972, respectively. In one embodiment the selectable marker gene is a drug selectable marker, such as the PAC gene which confers resistance to puromycin, or a similar selectable marker gene that permits selection of cells that express genes characteristic of differentiated SMC.

In one embodiment, pluripotential cells such as somatic stem cells, P19 embryonal carcinoma cells or embryonic stem cells are stably transfected with a gene construct comprising a selectable marker gene operably linked to either the SM α-actin promoter or the SM myosin heavy chain promoter and a second selectable marker gene that is operably linked to a constitutive promoter. Cells that have been stably transfected will be identified by selecting for the second selectable marker and isolating those cells that express the second selectable marker. This population of stably transfected cells will then be screened for cells that express the first selectable marker to identify SMC progenitor cells.

In accordance with one embodiment, pluripotential cells such as somatic stem cells, P19 embryonal carcinoma cells or embryonic stem cells are transfected with a drug selectable marker gene such as SM α-actin-PAC, SM MHC-PAC, or a similar selectable marker gene that permits selection of cells that express genes characteristic of differentiated SMC. The cells are co-transfected with a marker gene such as hygromycin that permits drug selection of cells that have been stably transfected. Preferably, the cells are co-transfected using a single DNA construct that comprises both the selectable marker for the stably transfected cells (hygromycin, for example) and the selectable marker for selecting SMC progenitor cells (SM α-actin-puromycin or SM MHC-puromycin, for example). Multiple clones that survive selection with hygromycin (or similar marker) are then selected and these cells are amplified and optionally stored by freezing aliquots of the cells.

Aliquots of each line of hygromycin resistant cells are then screened by inducing conversion to SMC lineages and selecting for puromycin resistant cell lines. Several methods for conversion of SMC progenitor cells into SMC lineages have been previously described and vary depending on the type of pluripotential cell line employed (Drab et al., Faseb Journal 11:905-915 (1997); Hirschi et al., The Journal of Cell Biology 141(3), 805-814. 1998; Shah et al., Cell 85:331-343 and Blank et al., Circulation Research 76:742-749. For P19 cells this involves treatment of monolayer cultures with retinoic acid, whereas for ES cells this involves aggregation of ES cells into embryoid bodies followed by treatment with retinoic acid plus dibutyryl cAMP Cells that survive puromycin selection are selected and screened for expression of multiple SMC marker genes such as SM α-actin, SM MHC, h1-calponin, smoothelin, etc. using RT-PCR and immunohistochemical staining techniques using standard techniques as described in the Examples.

Cell lines that show high rates (i.e. >90%) and efficacy (i.e. high level expression of multiple SMC marker genes) of induction of SMC lineages are selected. These represent SMC progenitor cells (i.e. pluripotential cells that are capable of forming SMC lineages upon treatment with an appropriate defined stimulus). A unique advantage of the present invention is that it permits generation of pure populations of fully differentiated SMC that show contractile properties similar to SMC in vivo. No previous described methods have this capability.

The SMC progenitor cells isolated in accordance with the present invention and compositions comprising those cells are also encompassed by the present invention. In particular, the present invention is directed to a purified population of SMC progenitor cells, wherein >80% of the total cells express SM α-actin by 4 days following RA treatment. In one embodiment the SMC progenitor cell of the present invention comprises a recombinant gene construct comprising the SM α-actin or SM-MHC promoter operably linked to a selectable marker. In one embodiment the SMC progenitor cell comprises a stably integrated SM α-actin promoter-selectable marker gene, and more particularly the selectable marker is a puromycin resistance gene. The high efficacy of SMC differentiation observed with A404 cells is in marked contrast with that seen with parental P19 cells where <1-5% of cells were estimated to differentiate into SMCs within 4 days.

The SMC progenitor cells isolated in accordance with the present invention are used in accordance with one embodiment to identify and isolate additional marker proteins, genes, cell surface antigens, monoclonal or polyclonal antibodies, or other reagents that could be used for screening and/or isolation/purification of SMC progenitor cells in humans. For example, a differential gene array or proteomic analysis of A404 cells versus parental P19 cells could be performed to identify specific marker proteins expressed on the surface of SMC progenitor cells. One could then develop antibodies to that marker protein as a means of identifying and purifying (by antibody-based cell sorting methods) SMC progenitor cell populations.

In one embodiment the SMC progenitor cells are used to screen for markers that can be used to distinguish them from the multipotential cells from which they were derived. A variety of standard methods can be employed including gene expression profiling, proteomic analyses, and production of monoclonal antibodies that are specific for SMC progenitor cells. The former would involve expression profiling SMC progenitor cells versus parental cells and identifying genes unique to the SMC progenitor population. Proteomic screening might involve high throughput mixed peptide mass spec comparison of membrane preparations of parental versus SMC progenitor cells. Production of SMC progenitor cell monoclonal antibodies would involve immunizing mice with SMC progenitor cells or derivatives thereof (i.e. a membrane fraction), and subsequent production and screening for monoclonal antibodies that distinguish SMC progenitor cells versus parental multipotential cells.

The SMC progenitor cell reagents and markers identified by the methods of the present invention are then used in accordance with the present invention to identify and/or purify SMC progenitor cells from human tissue samples, embryonic stem cell populations, or other tissue sources of multipotential cells. For example, one might use antibodies specific for SMC progenitor cells in conjunction with a fluorescence activated cell sorter or other antibody based cell sorting method to identify and purify these cells from multipotential cells or tissues.

In one embodiment of the present invention the SMC progenitor cells are use to promote vascular development during in vitro or in vivo organogenesis. The availability of SMC progenitor cell populations may also have broad applications for the treatment of a wide variety of clinical diseases and syndromes in man that require SMC tissues or SMC containing organs. For example, the availability of replacement blood vessels would have broad utility in the cardiovascular field for bypass surgery, replacement of vessels damaged by trauma or disease, augmentation of atherosclerotic lesions judged to be at high risk for rupture of the fibrous cap, expression of a growth inhibitory factor/gene, expression of a coronary vasodilator, etc. Similarly, SMC tissues might be used for bladder augmentation surgery as a treatment for incontinence or bladder failure, for replacement/augmentation of gastrointestinal SMC, and other organs whose function relies in part on smooth muscle tissue function.

### EXAMPLE 1

Little is known regarding transcriptional regulatory mechanisms that control sequential and coordinate expression of genes during smooth muscle cell (SMC) differentiation. To facilitate mechanistic studies of SMC differentiation, a novel P19-derived clonal cell line (designated A404) harboring a SM α-actin promoter/intron-driven puromycin resistance gene was established. Retinoic acid plus puromycin treatment stimulated differentiation of multipotential A404 cells into SMCs that expressed multiple SMC differentiation marker genes including the definitive SM-lineage marker, SM myosin heavy chain. Various transcription factors were demonstrated to be upregulated coincidentally with expression of SMC differentiation marker genes through the use of this system.

Of interest, expression of SRF, whose function is critical for SMC-specific transcription, was high in undifferentiated A404 cells, and did not increase over the course of differentiation. However, chromatin immunoprecipitation analyses showed that SRF did not bind the target sites of endogenous SMC marker genes in chromatin in undifferentiated cells, but did in differentiated A404 cells, and was associated with hyperacetylation of histones H3 and H4. The present invention defines a novel cell system for studies of transcriptional regulation during the early stages of SMC differentiation, and using this system evidence was obtained for involvement of chromatin remodeling and selective recruitment of SRF to CArG elements in the induction of cell selective marker genes during SMC differentiation.

Materials and Methods

SM-specific Promoter-puromycin Resistance Gene Constructs and Selection of Stable Lines

The puromycin-N-acetyltransferase (PAC) gene was PCR amplified from a template DNA pIRESpuro2 (Clontech). The LacZ gene of pAUG β-gal (a generous gift of Dr. Eric Olson) was replaced with the PAC gene. Subsequently, either the SM α-actin promoter/intron (−2560 to +2784 bp) or the SM-MHC promoter/intron (−4200 to +11600 bp) was subcloned into the plasmid (SMA-PAC and MHC-PAC). To make the cytomegarovirus promoter-hygromycin resistance gene construct (pCMV-hyg), pIREShyg vector (Clontech) was digested with HindIII and ligated.

P19 cells were obtained from American Type Culture Collection (CRL-1825). Cells were maintained in α-minimum essential medium (α-MEM, Sigma, M0644) supplemented with 7.5% fetal bovine serum (FBS), 200 μg/ml L-glutamine and penicillin/streptomycin (Lifetechnologies). For transfection and differentiation induction experiments, P19 cell cultures less than 6 passages from the initial culture obtained from ATCC were used. For cloning of stable cell lines, linearized puromycin resistance genes and pCMV-hyg were transfected using either Superfect or Effectane (Qiagen). Clonal lines were selected by treatment with 200-400 μg/ml of Hygromycin B (Lifetechnologies) and maintained in α-MEM with 200 μg/ml Hygromycin B. Integrated puromycin resistance genes were detected by genomic PCR. The cell lines containing the resistance gene were further characterized for their ability to differentiate into SMCs as well as for PAC expression.

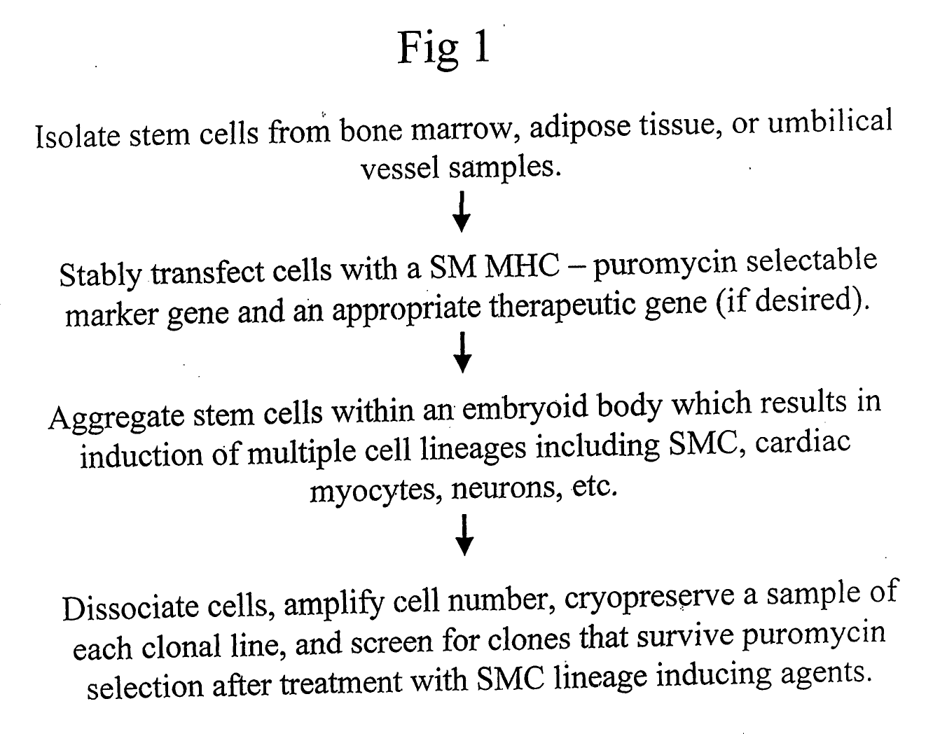

The culture methods for SMC differentiation are outlined in FIG. 1. Cells were trypsinized and plated in a 10 cm dish in α-MEM containing 7.5% FBS and 1 μmol/L all trans-retinoic acid (Sigma, R2625) at a density of 10,000 cells/cm² (day 0). The culture medium was replaced once on day 2. On day 3, RA was removed from the culture medium. On day 4, cells were trypsinized and plated in two 10 cm dishes in the medium containing 0.5 ng/ml puromycin (Clontech). Except otherwise noted, samples for various analyses were prepared from cells treated with puromycin for two days. During puromycin selection, the medium was replaced every day.

Reverse Transcriptase-PCR (RT-PCR)

For purification of RNA, mouse tissues were dissected and fat was removed from the tissues. RNA was purified from the whole aorta, SMC layers of the stomach and bladder, left and right ventricles of the heart, a portion of the liver, and a portion of cerebrum. Total RNA was purified using RNeasy mini kit (Qiagen). One μg of total RNA was reverse transcribed using Powerscript reverse transcriptase (Clontech) in a 20-μl reaction volume. For PCR amplification 1 μl of reverse transcribed samples was used. Quantitative multiplex PCR was performed with a gene-specific primer set and a QuantumRNA 18s internal standard primer set (Ambion) in a single tube. This internal standard primer set allows comparison between signals of target genes and highly abundant signals for the 18s internal standard by specifically reducing efficiency of amplification of the 18s standard. Linear amplification ranges for SM-MHC and SM α-actin were determined by taking PCR samples at various cycles and plotting amplification curves. Furthermore, in the conditions used for PCR, amplified signals of SM-MHC and SM α-actin were proportional to the amount of cDNA subjected to the PCR reactions. Although the strict linear amplification ranges for other genes were not determine, the signals did not plateau based on comparison of samples amplified with different numbers of PCR cycles. Therefore, PCR analyses were at least “semi-quantitative” and, thus, the results of PCR can be used for comparison of relative abundance of transcripts. Expression patterns of genes examined by RT-PCR in mouse tissues were consistent with reported tissue distributions. PCR products were resolved in 1.5-2% agarose gels and analyzed with ethidium bromide staining.

Electrophoretic Mobility Shift Assay (EMSA)

Nuclear extracts were prepared from undifferentiated A404 cells and differentiated A404 cells (day 7) using NE-PER Nuclear and Cytoplasmic Extraction Reagents (Pierce). Disruption of cell membranes was confirmed by microscopic observation prior to extraction of nuclear proteins. EMSAs were performed as previously described in Manabe et al., Biochem Biophys Res Commun. 1997; 239:598-605 and Madsen et al., J. Biol Chem. 1997; 272:6332-40.

Western Blotting and Immunocytochemistry

Western blot analysis was performed as previously described Regan et al., J. Clin Invest. 2000; 106:1139-47. SM-MHC expression was assessed using a rabbit anti-chicken SM-MHC antibody (1:200,000, a gift from Dr. U. Groschel-Stewart). The specificity of this antibody for mouse MHC isoforms has been thoroughly characterized. The antibody was not reactive with nonmuscle MHCs in Western blotting. Immnunocytochemical staining was performed using Vectastain ABC-AP kit (Vector Laboratories). Antibodies and dilutions used were 1:1000 anti-SM α-actin antibody (Sigma), 1:500 anti-SM-MHC antibody, and 1:1000 anti-neuron-specific β-tubulin antibody (TUJ1, Berkeley Antibody Company).

Chromatin Immunoprecipitation (ChIP) Assay

Methods for formalin treatment and preparation of chromatin samples were described previously Manabe and Owens, Cir Res 88: 1127-1134 (2001). A 10 cm dish of subconfluent undifferentiated and differentiated A404 cells (day 7) was used. Methods for immunoprecipitation using anti-SRF antibody (Santa Cruz Biotechnologies) were described previously Manabe and Owens, Cir Res 88: 1127-1134 (2001). Immunoprecipitation using anti-acetylated histone H3 and H4 antibodies (Upstate Biotechnology) was preformed according to the supplier's protocol. Immunoprecipitated chromatin samples were reverse-crosslinked and purified. Purified DNA samples were dissolved in TE buffer. An aliquot of the formalin-fixed total input chromatin DNA was reverse-crosslinked and purified to be used as a positive control in PCR analyses. For PCR analyses, equal amounts of DNA prepared from undifferentiated and differentiated cells were used. For each primer set, PCR analyses were performed using the sample immunoprecipitated with no antibody, the sample immunoprecipitated with the specific antibody, and the diluted total input DNA (1:200 dilution for SRF antibody; 1:16, H3; 1:8, H4). Various numbers of PCR cycles (26 to 35 cycles) were performed. Importantly, the final yield of each PCR fragment was found to be proportional to the relative input amount of DNA under the conditions used for PCR analyses.

Results

Isolation of a Puromycin-selectable P19 Derived Clonal Cell Line that Showed High Efficacy Formation of a SMC Lineage

In order to circumvent low efficacy of SMC differentiation of P19 cells, P19 clones were isolated that could be selected by puromycin for SMC lineages. P19 cells were cotransfected with either a −2560 to +2784 SM α-actin promoter/puromycin- N-acetyltransferase (SMA-PAC) or a −4200 to +11600 SM-MHC promoter/PAC (MHC-PAC), and a CMV promoter driven hygromycin gene. Subsequently, cells were treated with hygromycin to select stable transformants. Thirteen and twenty five random colonies were isolated from cells transfected with the SMA-PAC and MHC-PAC constructs, respectively. Genomic PCR was done to determine if there was integration of the PAC genes. Clones containing PAC genes were then further tested for their ability to differentiate into SMCs. Ten SMA-PAC clones and 18 MHC-PAC clones were treated with RA and then treated with puromycin. Two SMA-PAC clones and one MHC-PAC clone survived the puromycin selection. These clones were examined for expression of SM α-actin and SM-MHC. One clone designated A404 showed high-level expression of both markers.

To isolate MHC-PAC clones capable of efficient SMC differentiation another round of screening was conducted. Ten clones of the MHC-PAC gene resembling undifferentiated A404 cells were treated with RA. Three clones survived the puromycin selection. However, expression of SM-MHC in these MHC-PAC lines treated with RA and puromycin was weaker than that in RA-treated A404 cells at the mRNA level. Because of very strong expression of the SM α-actin and SM-MHC genes observed in differentiated A404 cells, A404 cells were used for further studies.

Multipotential A404 Cells Derived from P19 Cells Showed Highly Efficient Conversion into SMCs When Treated with Retinoic Acid

Undifferentiated A404 cells grew exponentially and had a spindle shape similar to a subpopulation of parental P19 cells. Expression of SM α-actin and SM-MHC was not detected in undifferentiated A404 cells. The culture methods employed for inducing SMC differentiation are outlined in FIG. 1. Cells were treated with 1 mmol/L RA for 3 days and then cultured in the standard medium for one day without RA. On day 4 the majority of these cells expressed SM α-actin and SM-MHC. A minor population of cells was neuron-like. Of particular note, expression of all SM marker genes analyzed was much higher than that of parental P19 cells treated with RA.

By treating cells with puromycin at a concentration that could eliminate all undifferentiated A404 cells in two days, expression of SM marker genes was further increased. SM-MHC protein was also abundantly expressed in puromycin treated cells, while it was not detected in undifferentiated cells. Although both SM1 and SM2 were detected by RT-PCR, SM2 was not detected by Western analyses.

To assess the efficiency and efficacy of SMC-differentiation, immunocytochemical analyses were performed using anti-SM α-actin, anti-SM-MHC, and anti-neuron-specific tubulin (TUJ1) antibodies. Undifferentiated cells were not stained with these antibodies. By 4 days after RA treatment, the majority of cells were SMC-like and stained positive for α-actin. Most SMC-like cells were also stained positively with SM-MHC antibody. The majority of α-actin negative cells were neuron-like in morphology and comprised 10-20% of the total cell population. Approximately half of these neuron-like cells were stained positively with neuron-specific TUJ1 antibody. After 2-days of puromycin treatment, the fraction of neuron-like cells was decreased to 5-10%. Very few cells (<0.1%) were positive for TUJ1. All other cells were SMC-like and positive for both SM α-actin and SM-MHC. Five-days of puromycin treatment further decreased the number of neuronal cells to less than 5% and virtually no cells were stained positively for TUJ1. These data indicate that treatment with puromycin enriched for SM α-actin-positive cells. Consistent with this, the expression level of a basic helix-loop-helix (bHLH) transcription factor, NeuroD, and a microtubule-associated protein, MAP2C, declined during puromycin treatment, whereas in puromycin-nontreated cells expression of these neuronal markers was sustained.

In order to demonstrate the stability and reproducibility of induction of SMC differentiation in the A404 line consisted of two subpopulations that could only differentiate into either SM or neuronal lineages, 11 subclones from A404 cells were isolated by dilutional cloning. All 11 clones were able to differentiate into SMCs and neurons upon RA treatment. These results clearly rule out the possibility that the A404 cell line consists of two subsets of cells that have the ability to differentiate into SMCs or neuronal cells. Rather, results show that A404 cells are capable of differentiating into multiple cell lineages.

The SM α-actin promoter/intron regulatory sequence is activated in developing striated muscle cells in mouse embryos during development. As such, it is possible that puromycin selection of RA treated A404 cells might result in selection of cardiac and/or skeletal myocytes. However, consistent with previous studies of McBurney et al., J Cell Biol. 1982; 94:253-62 that showed very low efficacy of induction of skeletal or cardiac lineages in RA treated P19 cells, very weak expression of cardiac α-actin was observed in RA-treated A404 cells on day 4. Moreover, cardiac α-actin expression was decreased by puromycin selection. No expression of cardiac α-MHC, skeletal α-actin, and a cardiomyocyte-specific homeobox protein Nkx2-5 was detected by RT-PCR analyses. These data indicate that very few cells differentiated into cardiac muscle lineages by RA treatment and that puromycin treatment did not enrich for cardiomyocytes within this cell system.

Various Transcription Factors Implicated in Control of SMC Differentiation are Induced by RA in A404 Cells

A number of cis-elements have been identified to be important for control of SMC-specific genes. However, relatively little is known regarding transcription factors that regulate expression of these genes particularly during the early stages of formation of SMC lineages from multipotential cells. Transcription of the SMC-specific genes has been shown to be dependent on complex transcriptional regulatory modules that contain multiple transcription factor binding sites. For example, the SM-MHC gene has recently been shown to be differentially regulated by multiple regulatory modules in SMC-subtypes in vivo in transgenic mice Manabe and Owens, Cir Res 88: 1127-1134 (2001). As such, it is likely that induction of SMC differentiation marker genes is regulated by multiple signals and transcription factors.

To begin elucidating the circuitry of transcription factors that induce SMC marker genes during early stages of SMC differentiation, and to test the potential utility of A404 cells for studies of transcriptional regulation of SMC marker genes, a catalog of transcription factors implicated in control of SMC marker genes were analyzed. Various transcription factors were found to be differentially regulated during SMC differentiation of A404 cells. For example, a Krüppel-like zinc finger transcription factor BTEB2 (KLF5) that we and others found was important for transcriptional control of SMC marker genes including SM22α was induced on day 1. BTEB2 expression was also detected in SM tissues including the stomach and bladder. GATA6 was also induced on day 1 in A404 cells and remained elevated throughout the course of SMC differentiation. In contrast, GATA4 and 5 were expressed only transiently at early time points. Although these initial results are descriptive, they demonstrate that various transcription factors implicated in control of SMC differentiation are induced in the early stages of differentiation of A404 cells and most importantly prior to detectable upregulation of SMC differentiation markers. As such, the RA treated A404 cell system described here should have utility for studies of the transcriptional regulatory circuits that control cell specification and gene expression during the early stages of SMC differentiation.

Whereas SRF was Abundantly Expressed in Multipotential A404 Cells, Only Cells that Undergo RA-stimulated SMC Differentiation showed SRF Binding to CArG Containing SMC Genes within Chromatin

SRF-binding sites or CArG elements are crucial for transcription of virtually all SMC differentiation marker genes characterized to date including SM-MHC and SM α-actin. In chicken proepicardial cells, it has been reported that SRF was markedly upregulated during SMC differentiation in vitro. Moreover, in proepicardial cells, inhibition of SRF function resulted in reduction in expression of SMC differentiation marker genes. Similarly, expression of SRF and its binding to CArG elements of SM γ-actin coincide with upregulation of this gene during chicken gizzard development. These results and observations that SRF is highly expressed in developing muscle cells suggest that high-level expression of SRF may contribute to SMC-selective transcriptional control. However, as reported herein SRF expression was not increased during differentiation of A404 cells into SMCs, but rather was abundantly expressed in both undifferentiated and differentiated A404 cells. An alternative possibility is that the activity of SRF may be regulated at the translational and/or post-translational levels. To test if the CArG-binding activity of SRF was increased in association with SMC differentiation, EMSAs were performed using nuclear extracts prepared from undifferentiated and differentiated A404 cells. No increases in SRF binding activity were observed between nuclear extracts derived from undifferentiated versus differentiated A404 cells despite the fact that the differentiated cells showed marked increases in expression of multiple CArG-dependent SMC differentiation marker genes.

Although SRF was abundantly expressed and was active in binding to CArG elements in vitro, SRF might not be able to bind CArG elements of the endogenous SMC differentiation marker genes due to the “closed” state of nucleosomal target sites. To directly test this hypothesis chromatin immunoprecipitation assays (CHIP) were performed to detect binding of transcription factors to target sites in chromatin in living cells. Undifferentiated and differentiated A404 cells were treated with formalin, and cross-linked chromatin was subjected to chromatin immunoprecipitation using anti-SRF antibody. Neither SM α-actin nor SM-MHC CArG regions were amplified from anti-SRF chromatin immunoprecipitates derived from undifferentiated A404 cells, whereas the c-fos promoter, which has been previously reported to be constitutively occupied by SRF in cells, was highly enriched in the anti-SRF chromatin immunoprecipitates from undifferentiated A404 cells. In contrast, both α-actin and SM-MHC CArG regions were enriched in immunoprecipitates from differentiated A404 cell sample. The enrichment of SM-MHC CArG regions in differentiated A404 cells was highly selective in that no enrichment of these regions in immunoprecipitates from differentiated L6 rat skeletal muscle cells was observed. However, it has been previously reported that the CArG region of the skeletal actin promoter was bound by SRF within chromatin in L6 skeletal muscle cells. The SM-MHC proximal promoter region that contains a TATA-box and transcription start site but not a CArG element showed no amplification. Likewise, the amylase gene, which is not CArG dependent, showed no amplification. Results of these ChIP assays thus provide clear evidence showing that differentiation of multipotential A404 cells into SMCs is associated with increased SRF binding to the SM-MHC and SM α-actin CArG elements within intact chromatin in the absence of any detectable change in SRF expression or binding activity as measured using EMSAs.

To determine if activation of the endogenous SMC marker genes might involve chromatin remodeling, the structure of histones was investigated. The amino terminal tails of histones H3 and H4 are dominant players in chromatin fiber folding and are targets for various histone modification enzymes. In particular, it has been extensively documented that acetylation of histones H3 and H4 play a central role in chromatin remodeling. Thus ChIP analyses were also performed with anti-acetylated histone H3 and H4 antibodies. Results showed that acetylation of histone H4 was increased in differentiated A404 cells as compared with undifferentiated cells at CArG-containing regulatory regions of the SM α-actin and SM-MHC genes. This increase was seen in the CArG regions within the 5′-flanking region of the SM α-actin gene as well as within the 5′-flanking and first intronic regions of the SM-MHC gene. No increase in acetylation of H4 was observed in skeletal actin or amylase genes. Interestingly, the SM-MHC 5′-flanking CArG region also showed hyperacetylation of histone H3 but this was not observed at the α-actin 5′-CArG region or SM-MHC intronic CArG region. The SM-MHC transcription start site showed hyperacetylation of both histones H3 and H4. These results provide evidence for differential hyperacetylation of histones at the regulatory regions of SMC differentiation marker genes. Moreover, taken together with results of ChIP assay findings suggest that induction of CArG-containing SMC differentiation marker genes such as the SM (α-actin and SM-MHC genes during early SMC differentiation may be regulated at least in part by changes in chromatin structure mediated by histone acetylation. To our knowledge, these results are the first to provide evidence for a role of chromatin remodeling in control of SMC differentiation.

Discussion

Establishment of a Highly Efficient in Vitro SMC Differentiation System

Multipotential P19 cells have potential utility for various studies of SMC biology because of their ability to differentiate into SMCs following RA treatment. However, their utility for many studies has been greatly compromised by the low frequency of differentiation of wild-type P19 cells into SMC lineages. The present invention is directed to isolation of a derivative of the pluripotential P19 cells that showed extremely high efficacy of SMC differentiation. Unlike parental P19 cells or other P19 derivatives, the great majority of A404 cells underwent differentiation into SMCs within 4 days of RA treatment. Indeed, based on immunocytochemical analyses, >80% of the total A404 cell population stained positively for SM α-actin by 4 days following RA treatment.

A stably integrated SM α-actin promoter-puromycin gene permitted further enrichment of SMCs and following 2 to 5-days treatment with puromycin, >90% of cells stained positively for both SM α-actin and the definitive SMC lineage marker SM-MHC. The high efficacy of SMC differentiation observed with A404 cells is in marked contrast with that seen with parental P19 cells where <1-5% of cells were estimated to differentiate into SMCs within 4 days (Blank and Owens, unpublished observations). Indeed, in the present studies SM α-actin expression was barely detectable in RA treated P19 cells by RT-PCR analyses. Suzuki et al. used a clonal P19 derived cell line stably expressing antisense RNA against a transcription factor Brn-2, which is crucial for neuronal differentiation. Although the block of Brn-2 resulted in a higher abundance in SMCs as compared with the RA treated wild-type P19 cells, expression of SM α-actin and SM-MHC was observed only in later stages culture (day 8-20). In contrast, these markers are readily detectable within 4 days in RA treated A404 cells.

From a practical viewpoint, the A404 cell system appears to have several additional advantages over other SMC model systems that have been described including Monc-1 cells 2, chicken proepicardial cells 5, and 10T1/2 cells 4. Although these cell systems show efficient differentiation into SMCs with kinetics similar to that of A404 cells, they have several shortcomings. First, although Monc-1 cells appear to be efficiently differentiated into SMCs in M199 medium with a time course similar to that of A404 cells, culture of undifferentiated cells requires a specifically formulated medium supplemented with chicken embryo extracts. In contrast, A404 cells grow exponentially in a standard culture medium (α-MEM) supplemented with FBS and stay in the undifferentiated state. A simple addition of RA to the culture medium induces SMC differentiation consistently and reproducibly. Similar high-level expression of SM α-actin and SM-MHC in A404 cells treated with RA has been observed a number of times over long culture periods of undifferentiated A404 cells. Undifferentiated A404 cells can be cultured for at least three months without significant loss in ability for SMC-differentiation.

Secondly, another in vitro model system, primary chicken proepicardial cells, appears to be programmed for differentiation into SMC lineages and to undergo spontaneous differentiation in culture dishes. However, it is not possible to maintain and propagate undifferentiated proepicardial cells in culture and to systematically initiate SMC differentiation with a defined stimulus. Third, Hirschi et al. described a system whereby multipotential 10T1/2 cells could be induced to express multiple SMC markers by coculture with endothelial cells. Although Hirschi et al. also provided some evidence for that TGF-β was capable of inducing SMC markers in 10T1/2, there is some contention as to whether it induces the definite SMC marker SM-MHC in this system or other fibroblast-like cells. Therefore, it is questionable whether TGF-β alone can induce full SMC differentiation in 10T1/2 cells.

As such, although these in vitro SMC differentiation systems have been well defined, the ease of culture and consistency in SMC differentiation initiated by RA of A404 cells would be particularly beneficial in studies of molecular mechanisms of SMC differentiation. Indeed, the rapid induction of expression of SMC differentiation marker genes from undetectable to the very high-level during A404-cell differentiation and elimination of non-SM cells by puromycin treatment have allowed investigators for the first time to examine molecular mechanisms that control induction of endogenous SMC marker gene within chromatin during early stages of SMC differentiation.

In addition, although the present studies focused on the use of pluripotential embryonic carcinoma cells, the experimental strategy can be employed in a similar manner with virtually any pluripotential or totipotential stem cell population. Thus the protocol should have general utility for induction, isolation, and purification of differentiated SMC or SMC progenitor cells from multiple pluripotential stem cell systems including human.

Results of present studies provide the first evidence, to our knowledge, for a role of SMC-specific and developmentally regulated chromatin remodeling in induction of SMC-specific genes during SMC differentiation. A key question is to determine what kinds of transcription factors could initiate this chromatin remodeling. It is well established that transcriptional regulation involves the complex interplay of factors controlling chromatin structure and transcriptional activation, although how these factors are involved in cell lineage-specific gene activation during cellular differentiation is poorly understood. There is extensive evidence showing that a number of transcription factors interact with HATs and histone deacetylases (HDACs) and that this interaction is required for activation and suppression of transcription. However, very little is known regarding roles of histone modifying enzymes in SMC-selective transcriptional control. During skeletal myogenesis, the MyoD family transcription factors are known to bind HATs and are likely to play a key role in chromatin remodeling. It has also been shown that MEF2, which cooperatively regulates skeletal muscle-specific genes with MyoD, is bound by HDACs and release of suppression by HDACs is required for transactivation by MEF2 during skeletal muscle differentiation. Given some of the similarities in transcriptional controls between skeletal and smooth muscle cells (e.g., common utilization of CArG elements), it is interesting to speculate that similar mechanisms may function during SMC differentiation. Interestingly, multiple transcription factors that have been shown to interact with HATs and HDACs including MEF2C and GATA6 were induced prior to expression of SMC marker genes during A404 cell differentiation.

### EXAMPLE 2

Induction of SMC Lineages in Multipotential Embryonic Stem Cells within Embryoid Bodies Treated with Retinoic Acid Plus Dibutyryl (db) cAMP

Embryonic stem cells exhibit nearly unlimited renewal capacity while being able to maintain a pluripotential state and so possess tremendous potential in a wide variety of tissue engineering applications. Cultivation of ES cells in aggregates, known as embryoid bodies, is required in order for them to display their full differentiation capacity in vitro. As embryoid bodies, these cells recapitulate many of the events of early embryonic development, including development of the three embryonic germ layers and have the potential to form a wide variety of differentiated cell types. Specifically, this system displays many aspects of vascular development including blood island formation vasculogenesis and angiogenesis. Of relevance to this invention, Drab et al., Faseb Journal 11:905-915 (1997) have presented evidence for induction of SMC lineages in an retinoic acid+dibutyryl cAMP embryonic stem cell model. Applicants have conducted a series of studies in ES cells/embryoid bodies similar to those of Drab et al. and derived highly differentiated, contractile SMC. These cells were found to express multiple SMC specific marker genes based on immuno-staining, transfection with a SM MHC promoter-LacZ gene, RT-PCR analysis, and Western analyses. Moreover, the SMC appear to be in a highly differentiated contractile state as evidenced by their expression of the SM-2 isoform of SM-MHC, and the fact that areas of slow peristaltic smooth muscle-like contraction were observed, quite distinctly from the rapid regular contractions exhibited by cardiomyocytes which also form frequently in the differentiating embryoid bodies.

The critical limitation of the embryoid body system described by Drab et al., Faseb Journal 11:905-915 (1997) for possible commercial or therapeutic applications is that the frequency of conversion of stem cells to SMC is very low (2-5%), and the embryoid bodies produced contain a multitude of other contaminating cell types. However, as reported herein the combination of the present unique SMC specific promoter/enhancer marker gene strategy together with the embryoid body model of SMC differentiation allows derivation of purified or enriched populations of differentiated SMC or SMC progenitor cells from various pluripotential stem cell sources. Moreover, based on evidence in the literature showing the production of various cell lineages from human stem cell sources using similar embryoid body and other strategies, one of ordinary skill in the art would appreciate that the methodologies of the present invention are readily adaptable to successful use using human pluripotential or totipotential stem cells.

In brief, the method would involve stably transfecting human embryonic stem cells (e.g. from a person's own embryonic stem cells obtained from umbilical chord samples), or somatic stem cells from bone marrow adipose tissue or other source, with a G418 resistance plasmid and a construct in which a puromycin resistance gene (or other marker gene) is coupled to a smooth muscle specific promoter (e.g. SMαA or SM-MHC). Since these constructs have been used (as described in Example 1) to derive the A404 cell line, there should be no difficulty in generating similar human stem cell lines by G418 selection. The smooth muscle specific promoters have previously been shown to direct expression of LacZ in a smooth muscle specific pattern in vivo (Madsen et al., Circ.Res. 82:908-917 (1998)) as well as in SMC derived in vitro from stably transfected ES cells.

The SM promoter-puromycin stem cell lines will be used to produce embryoid bodies according to the following protocol: ES cells are aggregated in hanging drop cultures (d0-d2) to form embryoid bodies. These are cultured in suspension (d2-d6) and allowed to differentiate on gelatin-coated dishes (d6+) under RA and db cAMP stimulation. However, at a variety of time points prior to the development of differentiated SMC (d2, d5, d7, d10) the embryoid bodies will be disaggregated by digestion with collagenase/dispase and the resulting single cell suspension plated at clonal density. After 48 hrs, colonies derived from single cells will be selected and trypsinized. Half the colony will be frozen down while the other half will be re-plated on gelatin coated wells and treated with RA and db-cAMP. Differentiation into SMC will then be screened by puromycin selection since only cells expressing smooth muscle specific markers will be resistant to puromycin. Since a large number of colonies may need to be frozen down prior to puromycin selection, throughput will be maximized by trypsinizing and freezing colonies directly in a 96 well plate format. For lines that survive this screening procedure, the corresponding frozen undifferentiated cells will be obtained and those cells will be characterize in greater detail. Essentially, the population of undifferentiated cells will first be expanded and then RA/cAMP-mediated induction of SMC differentiation markers (e.g. SMaA, SM-MHC, calponin h1, smoothelin) will be examine as well as markers of non-SMC (NM-β actin and NM-MHC). Methods for assessment of these markers by immunocytochemistry and autoradiographic and western analyses are already well established. Immunostaining will be visualized by differential interference contrast microscopy and changes accurately assessed at the mRNA level by real time RT-PCR (Bio-Rad I-cycler). To assess whether given cell lines also have potential to differentiate into other cell lineages, the above techniques will also be use to assess markers of cardiac (cardiac α-actin and cardiac α-MHC) or neuronal (neuroD and MAP2C) lineages. Undifferentiated ES cells and multiple clones that did not survive puromycin selection will be used as negative controls and aortic extracts as a positive control.

The above methodology is anticipated to be successful in deriving precursor cells that are able to form SMC with high efficiency since the ‘proof of concept’ has already been demonstrated with the A404 studies (see Example 1) and the fact that mouse ES cells have the capacity of differentiate into SMC in response to RA and db cAMP. However, it may be necessary to modify the protocol to achieve high efficacy of formation of SMC lineages. As such, the present invention includes coverage of these methods which are obvious to one skilled in stem cell methodologies. For example, it may be necessary to perform screens using ES/embryoid body conditioned media to better fix SMC lineage during some of the culture manipulations. Alternative extracellular matrix coatings including laminin, and collagen IV (which have previously been shown to enhance differentiation of SMC in culture) may also need to be used as well as mitotically inactivated feeder cells (to encourage the survival and proliferation of the desired cell lines if cells grow very poorly at clonal densities).

One variation of the preceding protocol will be to produce fully differentiated stem cell derived SMC. In brief, the SMC will be allowed to develop fully within the embryoid bodies, and cells expressing a SM MHC—fluorescence marker gene (e.g. EGFP) will be purified using fluorescence activated cell sorting S (FACS). We already clearly shown the feasibility of this in that we have generated several ES cell lines that express LacZ under the control of the SM-MHC promoter. Since LacZ fluorescence may also be used for cell sorting,this marker gene could be used for obtaining a purified population of SMC for potential therapeutic applications as outlined elsewhere in this application.

1. A method of identifying smooth muscle progenitor cells, said method comprising the steps of
providing a population of cells comprising totipotent or pluripotent cells;
transfecting said population of cells with a nucleic acid sequence comprising a smooth muscle cell specific promoter/enhancer operably linked to a marker;
inducing said population of cells to become smooth muscle cells; and
identifying smooth muscle progenitor cells based on the expression of the marker.

2. The method of claim 1 wherein the smooth muscle cell specific promoter is selected from the group consisting of the smooth muscle α-actin, SM22, calponin, smoothelin, smooth muscle myosin heavy chain promoters and derivatives of smooth muscle α-actin and smooth muscle myosin heavy chain promoters that exhibit selective activity in subtypes of SMC.

3. The method of claim 2 wherein the nucleic acid sequence further comprises a constitutive promoter operably linked to a second marker.

4. The method of claim 2 wherein the step of inducing the cells to become smooth muscle cells comprises the step of contacting the cells with retinoic acid or other inducing agents.

5. The method of claim 1 wherein the step of inducing the cells comprises the step of forming an embryoid body.

6. The method of claim 5 wherein the cells of the embryoid body are dissociated at a developmental stage where the cells remain pluripotent, and individual cells are clonally propagated to generate pools of progenitor cells.

7. The method of claim 5 wherein the cells of the embryoid body are allowed to develop until smooth muscle cells are expressed.

8. The method of claim 7 wherein the cells of the embryoid body are contacted with retinoic acid or other inducing agents.

9. The method of claim 3 wherein the smooth muscle cell specific promoter is operably linked to a first selectable marker and the constitutive promoter is operably linked to a second selectable marker, wherein the first and second selectable markers are different.

10. A population of smooth muscle progenitor cells identified by the method of claim 1.

11. A purified population of smooth muscle progenitor cells, wherein greater than 60% of the cells are induced into the smooth muscle cell linage by contacting the cells with a composition comprising a smooth muscle inducing agent.

12. The cells of claim 11, wherein the composition consists essentially of retinoic acid.

13. The cells of claim 11, wherein the composition consists essentially of retinoic acid and dibutyryl cAMP.

14. A method of generating a greater than 95% pure smooth muscle cells from a population of totipotent or pluripotent cells, said method comprising the steps of
providing a population of cells comprising totipotent or pluripotent cells;
transfecting said population of cells with a nucleic acid sequence comprising a smooth muscle cell promoter/enhancer operably linked to a marker;
inducing said population of cells; and
isolating those cells that express the marker.

15. The method of claim 14 wherein the step of inducing the cells comprises the step of forming an embryoid body.

16. The method of claim 15 wherein the marker is a selectable marker, and the cells expressing the marker are identified by culturing the population of cells under conditions where only those cells expressing the marker survive.

17. The method of claim 16 wherein the selectable marker is an antibiotic resistance encoding sequence.

18. A method of identifying smooth muscle progenitor cells, said method comprising the steps of
providing a population of cells comprising totipotent or pluripotent cells;
transfecting said population of cells with a nucleic acid sequence comprising a smooth muscle promoter/enhancer element operably linked to a marker;
inducing said population of cells to differentiate;
isolating individual induced cells and propagating said isolated cells in the absence of further induction;
inducing the propagated cells to become smooth muscle cells;
identifying the progenitor cells that gave rise to smooth muscle cells at high efficiency.

19. The method of claim 18 wherein greater than 90% of the progenitor cells differentiate into smooth muscle cells.

20. The method of claim 18 wherein the step of inducing the cells comprises the step of forming an embryoid body.

21. The method of claim 20 wherein the cells of the embryoid body are dissociated at a developmental stage when the cells remain pluripotent, and individual cells are clonally propagated to generate pools of potential progenitor cells and a portion of each pool of potential progenitor cell is induced to determine if the cells will differentiate to smooth muscle cells at high efficiency.

22. A smooth muscle progenitor cell produced by the method of claim 18.
